(19) United States
(12) Patent Application Publication
(10) Pub. No.: US 2014/0223600 A1
(43) Pub. Date: Aug. 7, 2014

(54) Transgenic Camelina sativa plant having modified fatty acid contents of seed oil

(71) Applicant: Agragen, LLC, Cincinnati, OH (US)

(72) Inventors: Seppo Paavo Kaijalainen, Helsinki (FI); Kimmo Koivu, Itasalmi (FI); Viktor Kuvshinov, Vantaa (FI); Eric Murphy, Grand Forks, ND (US)

(73) Assignee: Agragen, LLC, Cincinnati, OH (US)

(21) Appl. No.: 13/986,461

(22) Filed: May 6, 2013

Related U.S. Application Data
(60) Provisional application No. 61/741,129, filed on Jul. 13, 2012.

(51) Int. Cl. C12N 15/82

(52) U.S. Cl. CPC C12N 15/8247
USPC 800/281; 800/298; 536/23.6

(57) ABSTRACT

This disclosure provides a method to modify seed oil composition of Camelina sativa plants. The disclosure also provides novel promoters and gene sequences for modification of plant seed oil composition.

### FIELD OF THE INVENTION

This invention relates to genetic engineering of oil contents of crop plants. More specifically this invention relates to modified fatty acid contents in Camelina sativa plants. The inventions relates further to novel promoter sequences.

### BACKGROUND OF THE INVENTION

Camelina sativa (L. Crantz) belongs to the family Brassicaceae in the tribe Sisymbrieae and both spring- and winter forms are in production. It is a low-input crop adapted to low fertility soils. Results from long-term experiments in Central Europe have shown that the seed yields of Camelina sativa are comparable to the yields of oil seed rape.

As Camelina sativa is a minor crop species, very little has been done in terms of its breeding aside from testing different accessions for agronomic traits and oil profiles. However, due to the high oil content of Camelina sativa seeds (varying between 30-40%), there has been a renewed interest in Camelina sativa oil. Camelina sativa seeds have high content of polyunsaturated fatty acids, about 50-60% with an excellent balance of useful fatty acids including 30-40% of alpha-linolenic acid, which is an omega-3 oil. Omega-3 oils from plants metabolically resemble marine omega-3 oils and are rarely found in other seed crops. Furthermore, Camelina sativa seeds contain high amount of tocopherols (appr. 600 ppm) with a unique oxidative stability. Moreover, there is an increasing interest in Camelina sativa as animal feed.

In addition, there is an impeding need to introduce commercial crops to provide vegetable oils for biofuel production without displacing food crops from rich soils. Because Camelina sativa is well suited to marginal soils, this plant species offers an alternative crop that can be grown and harvested in large quantities. However, because of limited breeding success, improvements in Camelina sativa are lacking.

There is a need for altered fatty acid compositions in oil plants. Camelina sativa oil is rich from 18 carbon fatty acids but does not have shorter carbon bodies, such as 12 carbons, in the fatty acid compositions. The instant invention resolves the existing problem by modifying Camelina sativa seed fatty acids and thereby providing a number of new uses for the seed oil.

### SHORT DESCRIPTION OF THE FIGURES

FIG. 1 depicts the fatty acid synthesis in plant cells.

FIG. 2 depicts an example of transformation constructs used. This construct contains Umbellularia califonica thioesterase under control of Brassica napus NapA-promoter and terminator from U. californica thioesterase.

FIG. 3 depicts an example of transformation constructs used. This construct contains Umbellularia californica thioesterasase under control of Brassica napus NapA promoter and U. californica thioesterase terminator. Cocos nutifera lysophosphatidic acid acyltransferasae (Cn-LPAT) is under control of Brassica napus NapA promoter and CN-LPAT termination.

### DESCRIPTION OF THE INVENTION

The present invention provides methods for producing Camelina plants and cultivars showing increased 12:0 and 14:0 fatty acid levels in the seed oil. Moreover, the present invention provides novel seed specific promoter and terminator, along with novel Camelina sativa thioesterase encoding gene for use of modification of fatty acid contents in plant seeds.

Camelina sativa seeds contain high levels of 18 carbon fatty acids, but no 12-carbon fatty acids. Table 1 below shows fatty acid analysis of seed oil of Camelina sativa.

Table 1 shows fatty acid analysis of seed oil of Camelina sativa grown on irrigated land in Yuma, Ariz. in winter 2005. The values represent mean+/−standard deviation for four separate analysis of oil expressed as mole %.

| Fatty Acid | Mean | SD | RSD |
|---|---|---|---|
| 16:0 | 5.7 | 0.1 | 1.8 |
| 18:0 | 2.5 | 0.1 | 2.4 |
| 18:1 n-9 | 15.5 | 0.0 | 0.3 |
| 18:2 n-6 | 16.8 | 0.1 | 0.6 |
| 18:3 n-3 | 39.0 | 0.2 | 0.5 |
| 20:0 | 0.1 | 0.0 | 0.0 |
| 20:1 n-9 | 14.7 | 0.2 | 1.5 |
| 20:2 n-6 | 1.8 | 0.1 | 4.5 |
| 22:0 | 1.3 | 0.1 | 3.9 |
| 22:1 n-9 | 2.4 | 0.1 | 3.9 |
| 24:0 | 0.3 | 0.0 | 18.2 |
| 24:1 n-9 | 0.1 | 0.0 | 0.0 |
| sat | 9.7 | 0.1 | 0.5 |
| unsat | 90.3 | 0.1 | 0.1 |
| MUFA | 32.7 | 0.2 | 0.7 |
| PUFA | 57.6 | 0.2 | 0.4 |
| n-3 | 36.5 | 5.0 | 13.8 |
| n-6 | 18.6 | 0.1 | 0.4 |

Lauric acid (dedecanoic acid; 12:0 fatty acid) is the main fatty acid in coconut oil and in palm kernel oil. It is a white, powdery solid with a faint odour of bay oil or soap. Lauric acid has a very low toxicity and so it is used in many soaps and shampoos. Sodium lauryl sulfate is the most common lauric-acid derived compound used for these purposes.

Because lauric acid has a non-polar hydrocarbon tail and a polar carboxylic acid head, it can interact with polar solvents as well as with fats allowing water to dissolve fats. Accordingly, lauric acid is a preferred product for detergent industry.

Other prospective industries for lauric acid and other short and medium chain fatty acids are biofuel industries. Because Camelina sativa is a low input plant that provides reasonable oil yields even in harsh environments, Camelina oil has high potential for biofuel industries. The fact however remains that the natural oil composition of Camelina sativa offers challenges for production of conventional biodiesel.

Because of the limited biodiversity of Camelina germplasm, this disclosure provides biotechnological means for modifying the oil composition of Camelina seeds toward higher contents of lauric acid and other medium chain fatty acids such as 14:0 fatty acids.

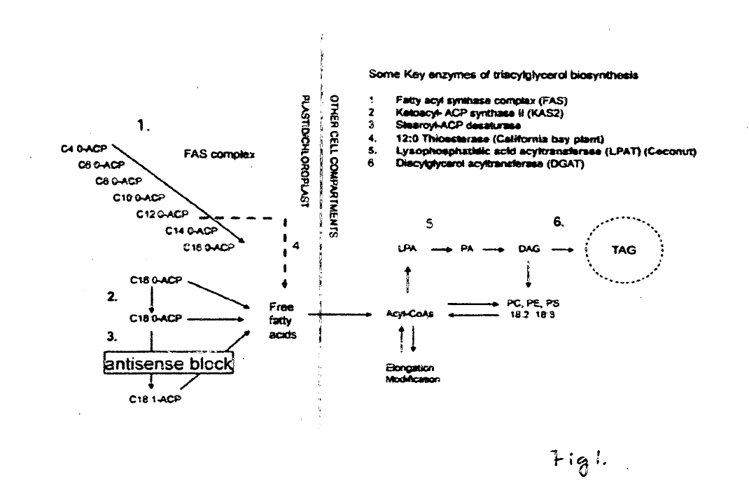

FIG. 1 depicts the fatty acid synthesis in plant cells. In natural conditions, fatty acids are synthesised with 16 carbon chain before releasing them to free fatty acid pool. Adding a thioesterase enzyme to the system would release the fatty acids already when there are only 12 carbon atoms in the chain and accordingly this would increase the amount of laureate acid in the seeds. Adding lysophosphatidic acid acyltransferase (LPAT) would allow the system to increase attachment of the free fatty acids into glycerol and thereby increase the amount of triacylglycerols. Furthermore, our goal was to decrease amount of unsaturated fatty acids, such as 18:1 fatty acid in order to keep the free fatty acid pool rich with medium length saturated fatty acids. To reach this goal we intend to block desaturation of 18:0 fatty acid by transforming the plants with a construct having antisense stearoyl-ACP desaturase.

Davies et al. (U.S. Pat. No. 5,344,771) transformed Brassica plants with DNA sequence encoding an Umbellularia californica C12:0 preferring acyl-ACP thioesterase under CaMV 35S promoter. The transgenic Brassica seed cells showed increased percentage of C12:0 fatty acids as compared to non transformed Brassica seed cells.

Davies et al (U.S. Pat. No. 5,563,058) purified coconut lysophosphatidic acid acyl transferase (LPAT).

High lauric acid canola was approved by the USDA for open field cultivation in 1994 and a significant commercial acreage was planted in ND and MN. High lauric acid canola had slightly lower yields and longer time to maturity as compared to non-GMO Canola.

This disclosure provides transgenic Camelina sativa plants with modified fatty acid composition in the seeds. This disclosure provides novel gene sequences to modify the fatty acid composition and novel methods to improve expression of the desired gene product.

This disclosure provides transgenic Camelina sativa plants that have been transformed by Agrobacterium mediated transformation with lauric acid-acyl carrier protein (ACP) (EC 3.1.2.21-dodecanoyl-(acyl-carrier-protein)hydrolase) from California bay plant (Umbellularia californica), lysophosphatidic acid acyltranferase (LPAT) (EC 2.3.1.51-1-acylglycerol-3-phospahte O-acyltransferase) from coconut endosperm and/or antisense construct of stearoyl-ACP desaturase of Camelina sativa (SEQ ID NO:6).

The invention is now described by means of non limiting examples. One skilled in the art will realize that many modifications can be made without diverting from the spirit of this invention.

### Example 1

### Camelina sativa Seed Storage Protein Regulatory Sequences

cDNA clones representing m-RNA populations of developing Camelina sativa seeds were sequenced. Based on most abundant sequence (Protein-28), the regions around the coding sequence were cloned using Genome Walking techniques and inverse-PCR. The coding region is preceded by promoter P-Cs28L (SEQ ID NO: 1) and followed by terminator T-Cs28 (SEQ ID NO:2). The sequences of the promoter and the terminator are shown below.

P-Cs28L (SEQ ID NO: 1):
CATATGAGAATAGCATACAGTGCTATTTTTTCTATAAATGATGACATGCCATTATCGGC
TATACTATAAATAGAGTTTTCAGATTCAATCATTAAATTCGTGAATAATATTTGAAAATT
GATTTAAGATTATCTCCTATATATTAATAGAGAAGCACACTTGAGAAAAAAGCTGATGT
GTCAGCGTTACAGAGTTCAGAACACTTTTTATCAAATAATTCTCAAAACATCTACTTATT
TACAACTCCCTGCCATTGTATTTATTAAAAAAAAAAAAAAAATCTATAATCCTCTCCTCT
CATCTCATCATTATTTACATATATATCATTGACATATATAAGACAATGTTATTTTCTATAA
GTTTTTAAAATAAAAAATTTAATCAACAATTAAATCCAGAAATGTATTTAATTATCAAATT
TATAACATATTTAATTATTAGAAATAAATAATATTTCACAAACAATAAAAAAATATTYATT
TATTTACCATTTTTATACATTTTTCTCATTGCATTTTTAAACTTATGATTTGTTTAAATTAA
ATCGATTAATCTAAAAAGTATTTTTTATCTATTTAATGGTATAGTGATGTAGATGATAAT
GTGTAAAATATGTGAATTTGATTTTTAGAAACACAAAAACAAATCAAAAATTTCTACACC
ATCTTAAAATTCTTTTCCAAGTTCAAATATTTCACGGATAAAACGTATTTTACCGAAAGT
AAACGTAAATTTGAATAAACAAAAAAAAAACTTATTTTGTTTTACACAAAATAAATCTCA
AATCTCAAATAATAATTTTACTCATAATATTTTATTTAAATTGATTTATCCCGCACATAGT
GCGATTGGTACCTATTTAATTTATTGAGGACAATCGCATGTTACTTTTTTGTTATTAGGG
ATAATCCGGGTTGAAGCCTGGTTCATCTTCGGTCGATTGCACGTTCACCGGCCGAGTG
AGCTAATTGACGTAAAATGTGGCATTAAATAGAAGTTAATTAAAGAATTCGAATTGACA
TCATGCCCCGATACTTTAATTACTAGCTAGACACTCGCATGGTTACAAATTAAACACAA
TGTGATATATGCACATCAACTGAATACACACATACACTTTAGTAAAATTTCATAAATATA
TGCTAGAATTACAATTTTCAGTTTGTTTGAAGTCATTTAGCCACTTTACATATTATCG
AGCCTGTGATTTATATATCTAATTAATTTATTAACATTATTCAATGGGTTGTGTGAAATC
TTTTTTTTTTTTAATATCTACATTTCAGATGAACATAGTACCTAGCTAAAACGTAATTCTA
CTGATTCCAGTTTTAATATACCATACCAAAAGATTGACCTTATCATCTTACTATAATGGA
ATCAAATTACAACACAAGGCTTTTTCTTCTTTTATTAACCTTGCTTGTTCTATATCGTTC
ATAAGATGTCATGTCAGAACTTGAGCTACAGATCACATATAGCATGCAGACGCGGAGG
GCTGGTGTTGTTCGTCACTTGTCACTCCAACACCTAATCTCGACAACAACCTAAGCGC
TTCACTCTCTCGCACATACATGCATTCTTACACGTGATKGCCATGCAAATCTACTTTCT
CACCTATAAATACAAACCAACCTTTCACTACACTCTTCACTCAAACCAAAACAAGAAAC
CATACACAAATAGCAA
T-Cs28 (SEQ ID NO:2):
ATTCGAAACAAAACCCTCTAGCGTATGAGTGTGGTTGTTGATACATGTTAACATCACAC
TTCATAGTCTGCTTTATGAAACTGTAGCTTTAGGATGTTTGAGGCTAATGTAATTAGCG
CTACTCCTCAATAAATAAAAGTTTTGTTTATATGTATATATCAACTGCCATATGCTCTGT
ATAGGTGGTCTAGGATATNAGCTCTCAAGCAAATATCCCAATCACATTTGCGGGNTTA
CTTTATCAATCGAACTCATACATCGAGCCAAACACCATTAAAATTGCACTATGACTGTA
ATTATTAATTATATTTACGTTTCCCACAACCGAAGACATGGAGGATATATAGAGACGGT
GTGTTTTCATTGAAGACGGCAAAACTTCAACTAGTTATAGTTGTCATCTTATCAACTCA
GTATTAAACATTTATCCATAATATAATTAAAGAACATTTTTGCACGAACTCAATCAATAT
GTTAGTTAACTTTTCTTTTTTNAGCAGTCAGTGACTGAGTCGCACACATACTAGTTAAA
AATTAGGGNCTAGACGGTGACTACTCTCAAGGGTGAAAANTTTGTTNGCAAGAGTGTG
CCGCTACGAGAATGAAGCATCATGCATATGTGAATTNACAGCCTAAGTCCTATTACCA
CACCGCGCCACCAGGTACGGGTTAATTTACTATCGGCCTCAAGAAATTGCACGCCATC
AAGTGGAAGAGACAAGTCAAAAAGGAATTTTACATAACATGAAAGCGAAAAACAAAAA
TGATAAATTACGTGACATGACCTGTTTGACTAATAGTCGCTAACGTTTGTGGAAAAAGA
GTGATGCAATTATATAGCCTTTGTGGTCATTGGTCAATAGTGTAAAACGTTACTTAATA
AATAAACAGTGATAACAAAGGCTTATAAAGACTTGTAGATGTTGTTCTGTGATCACAAT
AGGTTCTTGTTAAGATCCGGTTTGATGAAGATTTCAGAAAGAGCCATTCGTTTGGTTTT
GTGAAGCTATTTTTTGGTTTAAGCTAAACGTGGTTAGGAAGTTAGTATATACTTAGTGA
T

### Example 2

### Stearoyl-ACP Desaturase (Cs-SACPD) of Camelina sativa Seeds

The sequence of Stearoyl-ACP desaturase encoding gene of Camelina sativa seeds was obtained by amplifying coding region of cDNA pool representing mRNA of developing Camelina seeds using homologous sequences of Brassica napus and Arabidopsis thaliana as primers. Based on the obtained sequences, primers were designed for amplification and cloning 5′ and 3′ ends of Cs-SACPD cDNA using cDNA ligated to intramolecular circular as a template.

The sequences of the 5′UTR (SEQ ID NO: 3), the coding sequence (CDS; SEQ ID NO:4) and of the 3′UTR (SEQ ID NO:5) are provided below. SEQ ID NO: 6 represents the antisense sequence of Cs-SACPD.

5′UTR (SEQ ID NO: 3):
ATTCTCTTTCTGTGGACGAAACTGAACCTGAGAACTAAAACAAAAAAGCCAGAGCCAA
ACCCAGACCGAGTGTTAGAGATTGAGATTGAGATTGAGAGAGAGCAATTTAGCGCTGT
AGCAAGTACGATTCCATTCAA
CDS (SEQ ID NO: 4):
ATGGCTCTAAAGCTTAACCCTTTGGTGGCATCTCAGCCTTACAAATTCCCTTCCTCGAC
TCGTCCGCCTATCTCTTCTTTCAGATCTCCCAAGTTCCTCTGCCTCGCTTCATCTTCTC
CGGCTCTCAGCTCCGGCGCCAAGGAGGTTGAGAGTTTGAAGAAGCCATTTACCCCAC
CTAGGGAAGTGCATGTTCAAGTCTTGCACTCCATGCCACCTCAAAAGATCGAGATCTT
CAAATCTATGGAAAACTGGGCCGAGGAGAATCTTCTGATTCATCTCAAGGATGTTGAG
AAGTCTTGGCAACCCCAGGATTTCTTGCCTGATCCTGCATCGGATGGGTTTGAAGATC
AGGTAAGAGAGTTAAGAGAGAGGGCTAGAGAGCTTCCTGATGATTACTTTGTTGTTTT
GGTCGGGGACATGATCACAGAAGAAGCACTTCCGACCTATCAAACTATGTTGAACACT
TTGGACGGAGTTAGGGATGAAACAGGTGCTAGTCCTACTTCATGGGCTATTTGGACAA
GAGCTTGGACTGCAGAGGAAAACCGACATGGTGATCTTCTGAACAAATACCTTTACTT
GTCTGGTCGTGTTGACATGAGGCAGATCGAAAAGACCATTCAGTACTTGATTGGATCC
GGAATGGATCCGCGGACAGAGAATAACCCCTACCTTGGCTTCATCTATACTTCATTCC
AAGAAAGAGCGACCTTCATCTCTCACGGAAACACAGCCCGCCAAGCCAAAGAGCATG
GTGACTTCAAACTAGCCCAAATATGTGGCACAATAGCTGCAGACGAGAAGCGTCACGA
AACAGCATACACGAAGATAGTTGAGAAGCTCTTTGAGATTGATCCTGATGGTACAGTC
ATGGCTTTTGCAGACATGATGAGAAAGAAAATCTCAATGCCTGCTCACTTGATGTACG
ATGGGCGCAACGACAACCTCTTTGACAACTTCTCATCCGTGGCTCAGAGGCTCGGTGT
TTACACTGCCAAAGACTACGCAGACATTCTTGAGTTTTTGGTTGGTAGGTGGAAAATTG
GGGACTTAACTGGGCTATCAGGTGAAGGAAACAAAGCACAAGACTATCTATGCGGGTT
GTCTCCAAGAATCAAGAGATTGGATGAGAGAGCTCAAGCAAGAGCCAAGAAAGGACC
CAAGATTCCTTTCAGCTGGATACATGACAGAGAAGTGCAGCTCTAA
3′UTR (SEQ ID NO: 5):
AAAGGACACAGACAAAAAAACCCTCTCCTCTCTCGGTTACTCATTTCATCAGTCTGCTC
TTGAAATTGGTGTAGATTACTATGGTTTCTTCTGATAATGTTCGTGGGTCTACTAGTTTA
CAAAGTTGAGAAGCAGTGATTTTAGTATCTTTGTTTTTCCCAGTCACTATATGTTTGGG
TCATTGGTCCCTTCTTAGTACACTTTTGTAGTAGTTAAAACAGTTGAAGTCTGGTCTGT
ACTCAGTTTTCTCTGTGGAGTTTTGTTTGCAGTTCAGGTTAGTTTTGTTTGCAGTCTCT
CCGRAGGTTTCTTCNTGTTTTTNTTAGACAANCAACNAACAACTCATGNTGGCNTTTTT
AGCAATTTTGATAATCATAATGAATMTCNTTCCT
Antisense (Cs-AS-SACPD)
(SEQ ID NO: 6)
GAGCTGCACTTCTCTGTCATGTATCCAGCTGAAAGGAATCTTGGGTCCTTTCTTGGCT
CTTGCTTGAGCTCTCTCATCCAATCTCTTGATTCTTGGAGACAACCCGCATAGATAGTC
TTGTGCTTTGTTTCCTTCACCTGATAGCCCAGTTAAGTCCCCAATTTTCCACCTACCAA
CCAAAAACTCAAGAATGTCTGCGTAGTCTTTGGCAGTGTAAACACCGAGCCTCTGAGC
CACGGATGAGAAGTTGTCAAAGAGGTTGTCGTTGCGCCCATCGTACATCAAGTGAGC
AGGCATTGAGATTTTCTTTCTCATCATGTCTGCAAAAGCCATGACTGTACCATCAGGAT
CAATCTCAAAGAGCTTCTCAACTATCTTCGTGTATGCTGTTTCGTGACGCTTCTCGTCT
GCAGCTATTGTGCCACATATTTGGGCTAGTTTGAAGTCACCATGCTCTTTGGCTTGGC
GGGCTGTGTTTCCGTGAGAGATGAAGGTCGCTCTTTCTTGGAATGAAGTATAGATGAA
GCCAAGGTAGGGGTTATTCTCTGTCCGCGGATCCATTCCGGATCCAATCAAGTACTGA
ATGGTCTTTTCGATCTGCCTCATGTCAACACGACCAGACAAGTAAAGGTATTTGTTCAG
AAGATCACCATGTCGGTTTTCCTCTGCAGTCCAAGCTCTTGTCCAAATAGCCCATGAA
GTAGGACTAGCACCTGTTTCATCCCTAACTCCGTCCAAAGTGTTCAACATAGTTTGATA
GGTCGGAAGTGCTTCTTCTGTGATCATGTCCCCGACCAAAACAACAAAGTAATCATCA
GGAAGCTCTCTAGCCCTCTCTCTTAACTCTCTTACCTGATCTTCAAACCCATCCGATGC
AGGATCAGGCAAGAAATCCTGGGGTTGCCAAGACTTCTCAACATCCTTGAGATGAATC
AGAAGATTCTCCTCGGCCCAGTTTTCCATAGATTTGAAGATCTCGATCTTTTGAGGTGG
CATGGAGTGCAAGACTTGAACATGCACTTCCCTAGGTGGGGTAAATGGCTTCTTCAAA
CTCTCAACCTCCTTGGCGCCGGAGCTGAGAGCCGGAGAAGATGAAGCGAGGCAGAG
GAACTTGGGAGATCTGAAAGAAGAGATAGGCGGACGAGTCGAGGAAGGGAATTTGTA
AGGCTGAGATGCCACCAAAGGGTTAAGC

### Example 3

### Design of Transformation Constructs

Several plant transformation vectors were constructed for Agrobacterium-mediated transformation as described in patent applications U.S. Ser. Nos. 10/416,091; 12/288,791 and 12/290,379, which are incorporated herein by reference.

Basic transformation vector contains pBin19 based binary vector body and T-DNA region containing resistance gene against acetolactate synthase (ALS) inhibiting herbicide as is disclosed in the U.S. provisional patent application number U.S. 61/268,716, which is incorporated herein by reference. Alternatively transformation vector did not contain ALS resistance gene.

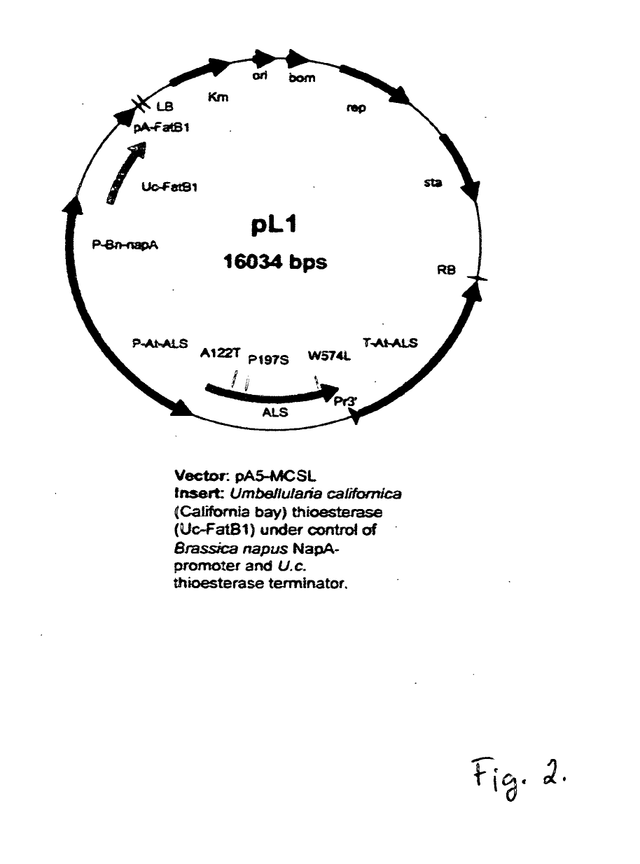

Synthesized gene encoding 12:0-ACP thioesterase and 3′-untranslated region was obtained from Geneart AG, Germany. 12:0-ACP thioesterase coding region and 3′ untranslated region were linked to a strong seed specific storage protein promoter. Brassica napus napin promoter and Camelina sativa P-Cs28L (SEQ ID NO: 1) were used in the constructs. FIG. 2 depicts an example of transformation constructs used.

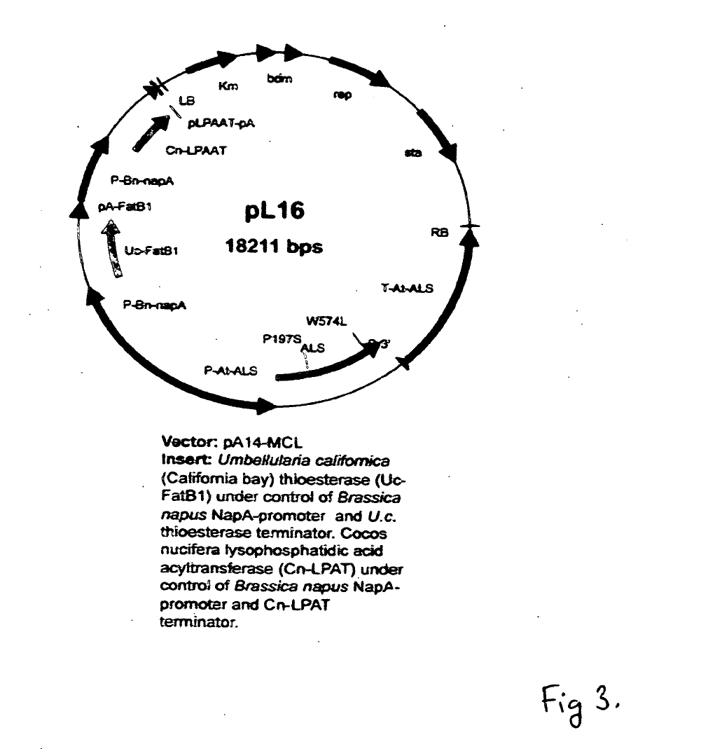

A more complex two enzymes containing construct was designed to efficiently synthesize and esterify lauric acid into oil bodies of the seeds. In addition to 12:0-ACP thioesterase, a synthetic gene encoding LPAT (Geneart AG, Germany) was used. LPAT aids in esterification of lauric acid into oil bodies by attaching lauric acid to lysophosphatidic acid (see FIG. 1). FIG. 3 shows an exemplary construct where both genes are expressed under napin storage protein promoter. Camelina storage protein promoter according SEQ ID NO: 1 was also used to direct the expression of the genes.

We also made constructs containing 12:0 thioesterase and antisense Stearoyl-ACP of Camelina sativa (SEQ ID NO: 6). A construct containing only the antisense sequence is also to be used in order to increase 16:0 and 18:0 acids which are suitable for biofuel industry. The genes may be under P-Cs28L promoter (SEQ ID NO: 1) or under Brassica napus napin promoter NapA.

### Example 4

### Bridging Sequence Between Simultaneously Expressed Multiple Genes

In constructs containing more than one coding gene sequence we have occasionally used a long DNA sequence in between of the coding sequences to separate them physically and to enable their independent expression. We also used shorter DNA elements that were expected to stop RNA-synthesis but those shorter sequences did not function as expected.

Plant RNA-polymerase reads a very long sequence of the preRNA and this is later shortened. Therefore RNA-polymerase reads the sequence far beyond the coding sequence of the gene and if the second gene is right after the first one there will be interference due to overlapping reading. The latter of the genes will interfere the expression of the first of the genes. Our approach is to prevent this by adding a bridging or intergenic sequence long enough between the two genes.

Another option widely used is to have the genes to be read in opposite directions; i.e. promoters are inserted into the plasmid next to each others. We speculate here that adding the bridging or intergenic sequence in between the genes may be beneficial.

We have used the intergenic region of Rubisco genes of tomato (SEQ ID NO:7). Accordingly the sequence is naturally a bridging sequence. An optimal length for the bridging sequence is about 1000 bp or more.

TomIGR
(SEQ ID NO: 7)
CCCACGTAGTAATCCTATCAACCTTGAAGACTTCAATTTGATGAATAATTCTCCCTTGT
TCTCTGCGTGAAGTCGTCGTATTCTTCATACGCGTCTTTTTCTTCTTATAGAGTTCCTTT
TGCCTTCAGTCCTCAGATAAGGTAAGGAAGTTATTATTAAACAAGGATTCCCTTTTAAA
GTACAATCCTTATTATATACAACTTCCTTCCTTAATAATATATTTAAGGTTTTCCTTATTT
GTATCAACTTATACCTTTAATATATTATTTTTGGCTTTGACAAATAACTCTATTTTCTTGA
TTACTTGGCTAATCCATTTCATTTTACTCGATCTTGGCTTCTTTTGCTGCGTACATTTGC
TATTGATTATTTGTGCTTCTTGTCTATCATCAAAACATGAATTATCGATTCTATCATATTC
TATCAGCTAGCTAGCACCACAAACTTGGATTTGGCTTTAGATTACTTCACTCCAGCCAT
ACTCCATGGCAATGGCCTCATTGTATGCGCTGCTTAGAAATAGACCAATTTTAATTTGT
TGCTATTGTAGTCATATTTTAATTATACGATTATTTACACGAGGCAGTGCAGGGTTCGC
AAATTGATTTCATCTCTTAAAGTTTCTGTCTATAGTTGGAAAGAATAGCAGGACATTTTT
AGTACGTTTTTAAAGAAGCATATCCATTACTATCCACAGTTGAGAGTGTCATCCTAACT
TTCTTGTACTTTCCTGTTGAGGATATTATTAAACCTATTAATAAAGACGAGTGACTCTTC
TNGGGNTAATCTCACANNNNNNNNNNNNNNNNNNNTAAAAAGAACTGCCAATTCTTCG
CTGAAGCTATTCTGTTGAAGTTGTTTAACCATGAAAGGTTATGAAATGCTTCTCTTATTA
GTTCGGTCCCAAGTCCAAAACTCTCTACATGATCACAGAGTCATTCCCCTCAGGCAGC
TTGAAAAAGTATTGGTCAAAGTACGATAATGGCGTTGCTATTGATTTGGCGAGTAACAA
AAATTGGGGCAGGAAGATTCTTGAAGATTTGAATTTTCTTCATTGTCAGAGGCAGGCA
GAGTCTGGAAGGTTTGAACTTTCTTCATTGTCAGAGCCCTAAGATCGTCCATCGAGAC
ATCACGACAATGTGTTCATTCAGAGTGGTGGGAACTGGGAAGCGAGTTACGCTTGGA
GAATTTGGGTTGGCAGAAGAGATTCACTTGTTATGGTTCTTGAACTATCACTATACATT
TCATAACACCTGCACAATCAGCATAGCTGAATATCAATCAACAATTGAGAAAGAAGGG
AGTGACTTAAATATCACATCAGGATTGTGATGTAACCCAGCCTACTAGTACTTTGATTG
TGGAAATGACATAAATAAGCTTCAAACAATAATATTTTCCACGACCTCCACCCCACCAT
TATCAAGGACGGTGATGAGTTTTCAATTGTGAGCAATACCAAACTTTGCGAGCTCATG
AACATGGTTTTAATTCTCCATCTCATTGATCTACTTCTAATTCTACACAATGAAAGCTAT
TTACTCCAAAAATAAAGCTTCTTTTTCCGCTTGTCAACCTACATTTACAATTCAAACTAT
GCACTAATCGAATTCCCGCCCTAGCGGCCGCGAATTCACTAGTGATTTTTTCCGCTTG
TCAACCTACATTTACAATTCAAACTATGCACTGAAAAGTACTAGTAATGCATAATAGAT
GCTATTAAGTTTGATTGCAAAAAAGACGTACAATCATCAAATAAACATGCCTAACAATA
ATGACAATATTTTCAACTTCCAAACTTATGATAAGAAGATAAATCATAACCATTATGAAC
TGCAAATTACTATCATTCAAACAAATCCATGATATTGTAGCGCAAAGAAGACACACAAC
ATGTCAAATTGTTAGCTTTCTACTTTTTCCTGATTGATTAATATGGCCATCCATCGATCT
TTTATAAGGGACACAAACTTATTAGTGTTTCCTGTTGTGCATTTTATCCAACACAAAGAA
GTTCGGCTAATTGTAATGTTCCTGCAAAATCAGCCACACTTTATTCATTTGAGTCCCAC
TGGACAAAAGTCTGTCTGTATTACAGATTTTAAGTATGTATTATAAAAGTCAACCAATCA
GCCTTTAAACTTGAACCCTACTTCAGATCAGGCAATCACCCAACCAGTTTCAATAACAA
TCTTATCTAAGAATTCAGTTCCAAGAACAAACTTATCTGAAGAATAAATGTAAAAATACT
CGATGCTAAAAGAAACTGAAGTTAACGTCATCCTGTATGTGGTAATATATCGTATAGAT
ACTGTTCTAAAAAACCTGTTATTTGATGGGTGTTAGTTAATAGAAAAATTCGACTAACC
AGAATATCAAGGAGCATTTGAATTGCCGCGTTGCTTTTCAATTTCTTGCTTTTGTTTCTC
AGCAAGTTTATCCAAAGCAGCTTCCAGAGCATCCCGACCTCCAATAAGCAACCTGGTG
ATGACCTCTGGATCTCTCTCAAATTGTGCCTCCTCGAACTCTTTTCGGACATTTTCTCT
AAGAACATCCCGCCAGGGAATGCCTTGAGAGTTGGCCCACATGAAGAAGCGGGTTGC
GCGGATGACATCTCGGTAGAGACTTAGAGCCTCGCGTCGACTGCTAGTGAGTCGTTG
TCTATTTAGGAGCTCGTTCTCGTCATCATCAAGGTTCTTCTCCTTTTTGACCACATGTC
TATCCAATAGTTCCTCCATAGTGTCTGGACCATGGTGCAGGAGGCCATAGTGATGCAA
GAGCCATCTTGACTTGAAACTATGATCCAAAGCAGTTGAAAGATTTCGGAACTTTCGAA
TATTAGCATTCATCTGAAACCTCGACTTGACTGGTACAGAAAGAGAAACAAGACTTTAG
AGAAATCGTACTTCATCATATACTTCACACGAGAAACGCATGTAGATCAACATGAAGTG
AAAAATGGTCCAAGTTAAAAATAAACTTGTTAAGAAGGTCAGTAACATCCAAACAGAAA
GTCTTGCTTTTCTTAAGAATGCTATCAAACACAATAGCCAGAGAACAGAAGTGGTGCG
CATCTTCTGGTATGAGAGATACTACAACAGCAACAACAACATACAACATACCCGAGAA
ATCTCACAAAGTGGGGGTATGCCAGATACTACATGATTGGAATATATTCCAGCTGATTC
AATACTTTATACAGCAATGCACGACAGGAATAAAGATGAACAAAATCAAAAAAAAAAAA
GAACTTCTCTTTTTCCATTTGGGCGCGTAATGAAAGAGCTCCATGTGGAAGAATGGGA
GAACCCACATGCTTATTCCATTCAGTTTAATCAGAATTCAAGCATAATCAATTTGGAAA
AAGCATAACCAAAACAGTATAGAACAGAGAAAATAGATAAATTAGAAGACAGCAACAC
TATAAAAAGAACAATTTACTCTTCACCGGAACTTCTCCTAATCGAATTCCCGCGGCCTA
GTGATTGAACGGAAGAAGAATTGGAAAATAGTGTTTGGCAATTGCGGGTCGAAAAATG
GGTTAAAATGGCAATTGCGGGTAGAGAAGATGGGCCATAAATGGTTACAAAATAGATA
TGGGCTCAACATATTTTCTGGGCAGCCAATTTTAAAGGCATTTTCCTTTGAGGAAATAA
TTTCTTTGGACTTCAGAATATGAGTTGAAAGTAATAATTCTAATAATGAAATTAAACAAG
GATGATTAAATGGCAACAAAATGGAGTAATATGGATAATCAACGCAACTATATAGAGAA
AAAATAATAGCGCTACCATATACGAAAAATAGTAAAAAATTATAATAATGATTCAGAATA
AATTATTAATAACTAAAAAGCGTAAAGAAATAAATTAGAGAATAAGTGATACAAAATTG
GATGTTAATGGATACTTCTTATAATTGCTTAAAAGGAATACAAGATGGGAAATAATGTG
TTATTATTATTGATGTATAAAGAATTTGTACAATTTTTGTATCAATAAAGTTCCAAAAATA
ATCTTTAAAAAATAAAAGTACCCTTTTATGAACTTTTTATCAAATAAATGAAATCCAATAT
TAGCAAAACATTGATATTATTACTAAATATTTGTTAAATTAAAAAATATGTCATTTTATTT
TTTAACAGATATTTTTTAAAGTAAATGTTATAAATTACGAAAAAGGGATTAATGAGTATC
AAAACAGCCTAAATGGGAGGAGACAATAMCAGAAATTTGCTGTAGTAAGGTGGCTTAA
GTCATCATTTAATTTGATATTATAAAAATTCTAATTAGTTTATAGTCTTTCTTTTCCTCTT
TTGTTTGTCTTGTATGCTAAAAAAGGTATATTATATCTATAAATTATGTAGCATAATGAC
CACATCTGGCATCATCTTTACACAATTCACCTAAATATCTCAAGCGAAGTTTTGCCAAA
ACTGAAGAAAAGATTTGAACAACCTATCAAGTAACAAAAATCCCAAACAATATAGTCAT
CTATATTAAATCTTTTCAATTGAAGAAATTGTCAAAGACACATACCTCTATGAGTTTTTT
CATCAATTTTTTTTTCTTTTTTAAACTGTATTTTTAAAAAAATATTGAATAAAACATGTCC
TATTCATTAGTTTGGGAACTTTAAGATAAGGAGIGTGTAATTTCAGAGGCTATTAATTTT
GAAATGTCAAGAGCCACATAATCCAATGGTTATGGTTGCTCTTAGATGAGGTTATTGCT
TTAGGTGAAA

### Example 5

### Increased Lauric Acid Content in the Seeds of T1 Lines

Camelina sativa plants were transformed with constructs containing thioesterase gene of Umbellularia californica. Table 2 below shows fatty acid analysis of the seeds of T1 lines. We have similar results of seed of T2 lines. As can be seen, there is an increase in 12:0 and 14:0 fatty acid contents in all transformed plants containing the tioesterase gene. 12:0 content increased up to 23%, as compared to no 12:0 detectable in control seeds grown under same greenhouse conditions. In the highest 12:0 producing lines 14:0 was also increased from none detected to 4%. Accordingly, content of medium chain saturated fatty acids increased to 27%. At the same time 18:0 was reduced by 50%. Moreover, 18:1n fatty acid was reduced by over 60%, and 18:2n−6 by 25%. Surprisingly, the amount of 18:3n−3 amount is conserved in the transgenic seeds.

This data proves, that modifying the contents of the fatty acids of Camelina sativa seeds by increasing medium chain unsaturated fatty acids does not affect the content of polyunsaturated 18:C fatty acids. Consequently, the transformed Camelina sativa seeds do contain a very unique fatty acid composition useful for various industrial purposes.

TABLE 2
Example of Increased Lauric Acid Content in the seeds of T1 line

| Fatty acid | 1 | 2 | 4 | 5 | 6 | 7 | 8 | 9 | 10 | Control | Vector |
|---|---|---|---|---|---|---|---|---|---|---|---|
| Lauric 12:0 | 3.4 | 6.4 | [symbol] | [symbol] | 0.2 | [symbol] | [symbol] | 10.7 | 4.4 | [symbol] | [symbol] |
| Myristic 14:0 | 0.7 | 1.1 | [symbol] | [symbol] | 0.1 | [symbol] | [symbol] | 2.4 | 0.8 | [symbol] | [symbol] |
| Palmitic 16:0 | 6.0 | 6.0 | 7.0 | 4.3 | 4.8 | 6.2 | 5.6 | 5.6 | 5.6 | 5.8 | 6.3 |
| Stearic 18:0 | 3.4 | 2.8 | 2.1 | 2.3 | 2.9 | 2.8 | 2.5 | 2.5 | 3.2 | 4.0 | 4.4 |
| Oleic 18:1n-9 | 14.8 | 14.0 | 9.9 | 8.8 | 16.9 | 10.9 | 10.6 | 11.1 | 11.8 | 17.2 | 14.7 |
| Linoleic 18:2n-6 | 16.4 | 17.1 | 11.7 | 12.4 | 17.3 | 16.6 | 13.8 | 14.3 | 13.8 | 16.1 | 14.6 |
| Linoleic 18:3n-3 | 31.7 | 29.7 | 28.9 | 30.8 | 32.3 | 27.2 | 30.3 | 33.5 | 36.8 | 31.5 | 34.1 |
| Arachidic 20:0 | 1.9 | 2.0 | 1.8 | 2.0 | 1.9 | 2.5 | 2.3 | 2.2 | 2.7 | 2.0 | 2.4 |
| Eicosenoic 20:1n-9 | 12.6 | 12.9 | 8.1 | 8.7 | 13.8 | 10.2 | 9.7 | 9.4 | 12.4 | 13.6 | 12.8 |
| Eicosadienoic 20:2n-6 | 1.8 | 1.6 | 0.9 | 1.1 | 1.7 | 1.3 | 2.1 | 1.5 | 1.6 | 2.4 | 1.8 |
| Eicosatrienoic 20:3n-3 | 1.4 | 1.2 | 0.8 | 1.0 | 1.4 | 0.9 | 1.2 | 1.3 | 1.6 | 1.5 | 1.7 |
| Behenic 22:0 | 0.7 | 0.6 | 0.4 | 0.5 | 0.5 | 0.4 | 0.7 | 0.5 | 0.5 | 0.6 | 0.9 |
| Erucic 22:1n-9 | 3.5 | 3.4 | 2.7 | 3.0 | 3.2 | 3.2 | 3.5 | 3.5 | 3.0 | 3.1 | 4.3 |
| Lignoseric 24:0 | 0.4 | 0.3 | 0.3 | 0.3 | 0.6 | 0.5 | 0.2 | 0.3 | 0.3 | 0.3 | 0.3 |
| Nervonic 24:1n-9 | 1.1 | 0.9 | 0.7 | 0.8 | 1.1 | 0.9 | 1.0 | 1.2 | 1.1 | 1.3 | 0.9 |
| Camelina parent line | BC | BC | BC | BC | BC | BC | BC | BC | BC | BC | BC* |

[symbol]

indicates data missing or illegible when filed

What is claimed is:

1. A method to produce modified fatty acid content in Camelina sativa seeds, said method comprising the steps of:
a) transforming Camelina sativa plants with a DNA construct comprising at least one nucleotide sequence selected from the group consisting of a nucleotide sequence encoding thioesterase of Umbellularia californica, a nucleotide sequence encoding LPAT of coconut endosperm and a nucleotide sequence encoding Camelina sativa stearoyl-ACP desaturase in antisense orientation according to SEQ ID NO:6;
b) regenerating and growing transgenic plants;
c) collecting transgenic seeds.

2. The method of claim 1, wherein the nucleotide sequences are under control of Camelina sativa seed storage protein promoter of SEQ ID NO: 1.

3. The method of claim 1, wherein the DNA construct comprises more than one nucleotide sequences selected from the group consisting of a nucleotide sequence encoding thioesterase of Umbellularia californica, a nucleotide sequence encoding LPAT of coconut endosperm and a nucleotide sequence encoding Camelina sativa stearoyl-ACP desaturase in antisense orientation according to SEQ ID NO: 6; and a bridging sequence is inserted between the nucleotide sequences.

4. The method of claim 3, wherein the bridging sequence is according to SEQ ID NO:7.

5. A transgenic Camelina sativa plant for modified seed oil composition, said Camelina plant carrying nucleotide sequences encoding one or more nucleotide sequences selected from the group consisting of a nucleotide sequence encoding thioesterase of Umbellularia californica, a nucleotide sequence encoding LPAT of coconut endosperm, and a nucleotide sequence encoding Camelina sativa stearoyl-ACP desaturase in antisense orientation according to SEQ ID NO:6.

6. A transgenic Camelina sativa seed, said seed comprising a modified fatty acid composition of seed oil and said modified fatty acid composition being achieved by the method of claim 1, 2 or 3.

7. The transgenic Camelina sativa seed of claim 6, wherein the modified fatty acid composition of seed oil comprises increased amounts of C12:0 and C14:0 fatty acids.

8. The transgenic Camelina sativa seed of claim 7, wherein the modified fatty acid composition of seed oil further comprises conserved amounts of C18:3 fatty acids.

9. An isolated nucleotide sequence encoding a novel seed storage protein promoter according to SEQ ID NO: 1.

10. An isolated nucleotide sequence encoding stearoyl-ACP desaturase according to SEQ ID NO: 5.

11. A method to express multiple gene products from a DNA-construct, said method comprising a step of inserting into an expression vector a bridging sequence between sequences encoding the gene products.

12. The method of claim 11, wherein the bridging sequence is according to SEQ ID NO:7.


SEQUENCE LISTING (7 sequences)

SEQ ID NO: 1 - DNA, 1691 bp, Camelina sativa
catatgagaatagcatacagtgctattttttctataaatgatgacatgccattatcggctatactataaatagagttttcagattcaatcattaaattcgtgaataatatttgaaaattgatttaagattatctcctatatattaatagagaagcacacttgagaaaaaagctgatgtgtcagcgttacagagttcagaacactttttatcaaataattctcaaaacatctacttatttacaactccctgccattgtatttattaaaaaaaaaaaaaaaatctataatcctctcctctcatctcatcattatttacatatatatcattgacatatataagacaatgttattttctataagtttttaaaataaaaaatttaatcaacaattaaatccagaaatgtatttaattatcaaatttataacatatttaattattagaaataaataatatttcacaaacaataaaaaaatattyatttatttaccatttttatacatttttctcattgcatttttaaacttatgatttgtttaaattaaatcgattaatctaaaaagtattttttatctatttaatggtatagtgatgtagatgataatgtgtaaaatatgtgaatttgatttttagaaacacaaaaacaaatcaaaaatttctacaccatcttaaaattcttttccaagttcaaatatttcacggataaaacgtattttaccgaaagtaaacgtaaatttgaataaacaaaaaaaaaacttattttgttttacacaaaataaatctcaaatctcaaataataattttactcataatattttatttaaattgatttatcccgcacatagtgcgattggtacctatttaatttattgaggacaatcgcatgttacttttttgttattagggataatccgggttgaagcctggttcatcttcggtcgattgcacgttcaccggccgagtgagctaattgacgtaaaatgtggcattaaatagaagttaattaaagaattcgaattgacatcatgccccgatactttaattactagctagacactcgcatggttacaaattaaacacaatgtgatatatgcacatcaactgaatacacacatacactttagtaaaatttcataaatatatgctagaattacaattttcagtttgtttgaagtcatttttgccactttttcatattatcgagcctgtgatttatatatctaattaatttattaacattattcaatgggttgtgtgaaatcttttttttttttaatatctacatttcagatgaacatagtacctagctaaaacgtaattctactgattccagttttaatataccataccaaaagattgaccttatcatcttactataatggaatcaaattacaacacaaggctttttcttcttttattaaccttgcttgttctatatcgttcataagatgtcatgtcagaacttgagctacagatcacatatagcatgcagacgcggagggctggtgttgttcgtcacttgtcactccaacacctaatctcgacaacaacctaagcgcttcactctctcgcacatacatgcattcttacacgtgatkgccatgcaaatctactttctcacctataaatacaaaccaacctttcactacactcttcactcaaaccaaaacaagaaaccatacacaaatagcaa

SEQ ID NO: 2 - DNA, 1060 bp, Camelina sativa
attcgaaacaaaaccctctagcgtatgagtgtggttgttgatacatgttaacatcacacttcatagtctgctttatgaaactgtagctttaggatgtttgaggctaatgtaattagcgctactcctcaataaataaaagttttgtttatatgtatatatcaactgccatatgctctgtataggtggtctaggatataagctctcaagcaaatatcccaatcacatttgcgggattactttatcaatcgaactcatacatcgagccaaacaccattaaaattgcactatgactgtaattattaattatatttacgtttcccacaaccgaagacatggaggatatatagagacggtgtgttttcattgaagacggcaaaacttcaactagttatagttgtcatcttatcaactcagtattaaacatttatccataatataattaaagaacatttttgcacgaactcaatcaatatgttagttaacttttcttttttaagcagtcagtgactgagtcgcacacatactagttaaaaattagggactagacggtgactactctcaagggtgaaaaatttgttagcaagagtgtgccgctacgagaatgaagcatcatgcatatgtgaattaacagcctaagtcctattaccacaccgcgccaccaggtacgggttaatttactatcggcctcaagaaattgcacgccatcaagtggaagagacaagtcaaaaaggaattttacataacatgaaagcgaaaaacaaaaatgataaattacgtgacatgacctgtttgactaatagtcgctaacgtttgtggaaaaagagtgatgcaattatatagcctttgtggtcattggtcaatagtgtaaaacgttacttaataaataaacagtgataacaaaggcttataaagacttgtagatgttgttctgtgatcacaataggttcttgttaagatccggtttgatgaagatttcagaaagagccattcgtttggttttgtgaagctattttttggtttaagctaaacgtggttaggaagttagtatatacttagtgat

SEQ ID NO: 3 - DNA, 137 bp, Camelina sativa
attctctttctgtggacgaaactgaacctgagaactaaaacaaaaaagccagagccaaacccagaccgagtgttagagattgagattgagattgagagagagcaatttagcgctgtagcaagtacgattccattcaa

SEQ ID NO: 4 - DNA, 1206 bp, Camelina sativa
atggctctaaagcttaaccctttggtggcatctcagccttacaaattcccttcctcgactcgtccgcctatctcttctttcagatctcccaagttcctctgcctcgcttcatcttctccggctctcagctccggcgccaaggaggttgagagtttgaagaagccatttaccccacctagggaagtgcatgttcaagtcttgcactccatgccacctcaaaagatcgagatcttcaaatctatggaaaactgggccgaggagaatcttctgattcatctcaaggatgttgagaagtcttggcaaccccaggatttcttgcctgatcctgcatcggatgggtttgaagatcaggtaagagagttaagagagagggctagagagcttcctgatgattactttgttgttttggtcggggacatgatcacagaagaagcacttccgacctatcaaactatgttgaacactttggacggagttagggatgaaacaggtgctagtcctacttcatgggctatttggacaagagcttggactgcagaggaaaaccgacatggtgatcttctgaacaaatacctttacttgtctggtcgtgttgacatgaggcagatcgaaaagaccattcagtacttgattggatccggaatggatccgcggacagagaataacccctaccttggcttcatctatacttcattccaagaaagagcgaccttcatctctcacggaaacacagcccgccaagccaaagagcatggtgacttcaaactagcccaaatatgtggcacaatagctgcagacgagaagcgtcacgaaacagcatacacgaagatagttgagaagctctttgagattgatcctgatggtacagtcatggcttttgcagacatgatgagaaagaaaatctcaatgcctgctcacttgatgtacgatgggcgcaacgacaacctctttgacaacttctcatccgtggctcagaggctcggtgtttacactgccaaagactacgcagacattcttgagtttttggttggtaggtggaaaattggggacttaactgggctatcaggtgaaggaaacaaagcacaagactatctatgcgggttgtctccaagaatcaagagattggatgagagagctcaagcaagagccaagaaaggacccaagattcctttcagctggatacatgacagagaagtgcagctctaa

SEQ ID NO: 5 - DNA, 389 bp, Camelina sativa
  misc_feature 300: n = A,T,C or G
aaaggacacagacaaaaaaaccctctcctctctcggttactcatttcatcagtctgctcttgaaattggtgtagattactatggtttcttctgataatgttcgtgggtctactagtttacaaagttgagaagcagtgattttagtatctttgtttttcccagtcactatatgtttgggtcattggtcccttcttagtacacttttgtagtagttaaaacagttgaagtctggtctgtactcagttttctctgtggagttttgtttgcagttcaggttagttttgtttgcagtctctccgnaggtttcttcatgttttttttagacaaacaacaaacaactcatgttggcttttttagcaattttgataatcataatgaatmtctttcct

SEQ ID NO: 6 - DNA, 1192 bp, Camelina sativa
gagctgcacttctctgtcatgtatccagctgaaaggaatcttgggtcctttcttggctcttgcttgagctctctcatccaatctcttgattcttggagacaacccgcatagatagtcttgtgctttgtttccttcacctgatagcccagttaagtccccaattttccacctaccaaccaaaaactcaagaatgtctgcgtagtctttggcagtgtaaacaccgagcctctgagccacggatgagaagttgtcaaagaggttgtcgttgcgcccatcgtacatcaagtgagcaggcattgagattttctttctcatcatgtctgcaaaagccatgactgtaccatcaggatcaatctcaaagagcttctcaactatcttcgtgtatgctgtttcgtgacgcttctcgtctgcagctattgtgccacatatttgggctagtttgaagtcaccatgctctttggcttggcgggctgtgtttccgtgagagatgaaggtcgctctttcttggaatgaagtatagatgaagccaaggtaggggttattctctgtccgcggatccattccggatccaatcaagtactgaatggtcttttcgatctgcctcatgtcaacacgaccagacaagtaaaggtatttgttcagaagatcaccatgtcggttttcctctgcagtccaagctcttgtccaaatagcccatgaagtaggactagcacctgtttcatccctaactccgtccaaagtgttcaacatagtttgataggtcggaagtgcttcttctgtgatcatgtccccgaccaaaacaacaaagtaatcatcaggaagctctctagccctctctcttaactctcttacctgatcttcaaacccatccgatgcaggatcaggcaagaaatcctggggttgccaagacttctcaacatccttgagatgaatcagaagattctcctcggcccagttttccatagatttgaagatctcgatcttttgaggtggcatggagtgcaagacttgaacatgcacttccctaggtggggtaaatggcttcttcaaactctcaacctccttggcgccggagctgagagccggagaagatgaagcgaggcagaggaacttgggagatctgaaagaagagataggcggacgagtcgaggaagggaatttgtaaggctgagatgccaccaaagggttaagc

SEQ ID NO: 7 - DNA, 4743 bp, Lycopersicon esculentum
  misc_feature 779, 783, 794, 795, 796, 797, 798, 799, 800, 801, 802,803, 804, 805, 806, 807, 808, 809, 810, 811, 812: n = A,T,C or G
cccacgtagtaatcctatcaaccttgaagacttcaatttgatgaataattctcccttgttctctgcgtgaagtcgtcgtattcttcatacgcgtctttttcttcttatagagttccttttgccttcagtcctcagataaggtaaggaagttattattaaacaaggattcccttttaaagtacaatccttattatatacaacttccttccttaataatatatttaaggttttccttatttgtatcaacttatacctttaatatattatttttggctttgacaaataactctattttcttgattacttggctaatccatttcattttactcgatcttggcttcttttgctgcgtacatttgctattgattatttgtgcttcttgtctatcatcaaaacatgaattatcgattctatcatattctatcagctagctagcaccacaaacttggatttggctttagattacttcactccagccatactccatggcaatggcctcattgtatgcgctgcttagaaatagaccaattttaatttgttgctattgtagtcatattttaattatacgattatttacacgaggcagtgcagggttcgcaaattgatttcatctcttaaagtttctgtctatagttggaaagaatagcaggacatttttagtacgtttttaaagaagcatatccattactatccacagttgagagtgtcatcctaactttcttgtactttcctgttgaggatattattaaacctattaataaagacgagtgactcttctngggntaatctcacannnnnnnnnnnnnnnnnnntaaaaagaactgccaattcttcgctgaagctattctgttgaagttgtttaaccatgaaaggttatgaaatgcttctcttattagttcggtcccaagtccaaaactctctacatgatcacagagtcattcccctcaggcagcttgaaaaagtattggtcaaagtacgataatggcgttgctattgatttggcgagtaacaaaaattggggcaggaagattcttgaagatttgaattttcttcattgtcagaggcaggcagagtctggaaggtttgaactttcttcattgtcagagccctaagatcgtccatcgagacatcacgacaatgtgttcattcagagtggtgggaactgggaagcgagttacgcttggagaatttgggttggcagaagagattcacttgttatggttcttgaactatcactatacatttcataacacctgcacaatcagcatagctgaatatcaatcaacaattgagaaagaagggagtgacttaaatatcacatcaggattgtgatgtaacccagcctactagtactttgattgtggaaatgacataaataagcttcaaacaataatattttccacgacctccaccccaccattatcaaggacggtgatgagttttcaattgtgagcaataccaaactttgcgagctcatgaacatggttttaattctccatctcattgatctacttctaattctacacaatgaaagctatttactccaaaaataaagcttctttttccgcttgtcaacctacatttacaattcaaactatgcactaatcgaattcccgccctagcggccgcgaattcactagtgattttttccgcttgtcaacctacatttacaattcaaactatgcactgaaaagtactagtaatgcataatagatgctattaagtttgattgcaaaaaagacgtacaatcatcaaataaacatgcctaacaataatgacaatattttcaacttccaaacttatgataagaagataaatcataaccattatgaactgcaaattactatcattcaaacaaatccatgatattgtagcgcaaagaagacacacaacatgtcaaattgttagctttctactttttcctgattgattaatatggccatccatcgatcttttataagggacacaaacttattagtgtttcctgttgtgcattttatccaacacaaagaagttcggctaattgtaatgttcctgcaaaatcagccacactttattcatttgagtcccactggacaaaagtctgtctgtattacagattttaagtatgtattataaaagtcaaccaatcagcctttaaacttgaaccctacttcagatcaggcaatcacccaaccagtttcaataacaatcttatctaagaattcagttccaagaacaaacttatctgaagaataaatgtaaaaatactcgatgctaaaagaaactgaagttaacgtcatcctgtatgtggtaatatatcgtatagatactgttctaaaaaacctgttatttgatgggtgttagttaatagaaaaattcgactaaccagaatatcaaggagcatttgaattgccgcgttgcttttcaatttcttgcttttgtttctcagcaagtttatccaaagcagcttccagagcatcccgacctccaataagcaacctggtgatgacctctggatctctctcaaattgtgcctcctcgaactcttttcggacattttctctaagaacatcccgccagggaatgccttgagagttggcccacatgaagaagcgggttgcgcggatgacatctcggtagagacttagagcctcgcgtcgactgctagtgagtcgttgtctatttaggagctcgttctcgtcatcatcaaggttcttctcctttttgaccacatgtctatccaatagttcctccatagtgtctggaccatggtgcaggaggccatagtgatgcaagagccatcttgacttgaaactatgatccaaagcagttgaaagatttcggaactttcgaatattagcattcatctgaaacctcgacttgactggtacagaaagagaaacaagactttagagaaatcgtacttcatcatatacttcacacgagaaacgcatgtagatcaacatgaagtgaaaaatggtccaagttaaaaataaacttgttaagaaggtcagtaacatccaaacagaaagtcttgcttttcttaagaatgctatcaaacacaatagccagagaacagaagtggtgcgcatcttctggtatgagagatactacaacagcaacaacaacatacaacatacccgagaaatctcacaaagtgggggtatgccagatactacatgattggaatatattccagctgattcaatactttatacagcaatgcacgacaggaataaagatgaacaaaatcaaaaaaaaaaaagaacttctctttttccatttgggcgcgtaatgaaagagctccatgtggaagaatgggagaacccacatgcttattccattcagtttaatcagaattcaagcataatcaatttggaaaaagcataaccaaaacagtatagaacagagaaaatagataaattagaagacagcaacactataaaaagaacaatttactcttcaccggaacttctcctaatcgaattcccgcggcctagtgattgaacggaagaagaattggaaaatagtgtttggcaattgcgggtcgaaaaatgggttaaaatggcaattgcgggtagagaagatgggccataaatggttacaaaatagatatgggctcaacatattttctgggcagccaattttaaaggcattttcctttgaggaaataatttctttggacttcagaatatgagttgaaagtaataattctaataatgaaattaaacaaggatgattaaatggcaacaaaatggagtaatatggataatcaacgcaactatatagagaaaaaataatagcgctaccatatacgaaaaatagtaaaaaattataataatgattcagaataaattattaataactaaaaagcgtaaagaaataaattagagaataagtgatacaaaattggatgttaatggatacttcttataattgcttaaaaggaatacaagatgggaaataatgtgttattattattgatgtataaagaatttgtacaatttttgtatcaataaagttccaaaaataatctttaaaaaataaaagtacccttttatgaactttttatcaaataaatgaaatccaatattagcaaaacattgatattattactaaatatttgttaaattaaaaaatatgtcattttattttttaacagatattttttaaagtaaatgttataaattacgaaaaagggattaatgagtatcaaaacagcctaaatgggaggagacaatamcagaaatttgctgtagtaaggtggcttaagtcatcatttaatttgatattataaaaattctaattagtttatagtctttcttttcctcttttgtttgtcttgtatgctaaaaaaggtatattatatctataaattatgtagcataatgaccacatctggcatcatctttacacaattcacctaaatatctcaagcgaagttttgccaaaactgaagaaaagatttgaacaacctatcaagtaacaaaaatcccaaacaatatagtcatctatattaaatcttttcaattgaagaaattgtcaaagacacatacctctatgagttttttcatcaatttttttttcttttttaaactgtatttttaaaaaaatattgaataaaacatgtcctattcattagtttgggaactttaagataaggagtgtgtaatttcagaggctattaattttgaaatgtcaagagccacataatccaatggttatggttgctcttagatgaggttattgctttaggtgaaa